(19) United States
(12) Patent Application Publication
(10) Pub. No.: US 2019/0046961 A1
(43) Pub. Date: Feb. 14, 2019

(54) SUPPORTED CATALYST

(71) Applicant: University College Cardiff Consultants Ltd., Cardiff (GB)

(72) Inventors: Graham John HUTCHINGS, Northallerton (GB); Christopher Dean EVANS, Cardiff (GB); Stuart TAYLOR, Cardiff (GB); Jonathan Keith BARTLEY, Cardiff (GB); Simon Antoni Walter KONDRAT, Cardiff (GB)

(21) Appl. No.: 16/079,346

(22) PCT Filed: Feb. 23, 2017
(86) PCT No.: PCT/GB2017/050471

§ 371 Date: Aug. 23, 2018

(30) Foreign Application Priority Data
Feb. 24, 2016 (GB) 1603156.9

(51) Int. Cl. B01J 23/89; B01J 35/00; B01J 35/10

(52) U.S. Cl. CPC B01J 23/894; C07C 51/235; B01J 35/1014; B01J 35/006

(57) ABSTRACT

Described herein is a supported catalyst for a liquid-phase reaction, the supported catalyst comprising a perovskite support comprising A-site species and B-site species and a catalytic component on a surface of the perovskite support. Also described herein is a method for tuning the selectivity of a supported catalyst.

### TECHNICAL FIELD

The present invention is directed to supported catalysts, methods for forming supported catalysts, processes for tuning supported catalysts, processes for identifying supported catalysts having selectivity for a desired reaction product, methods for making a desired reaction product employing a supported catalyst and uses of supported catalysts. In particular, the present invention relates to supported catalysts, methods, processes and uses in which the supported catalyst comprises a perovskite support and a catalytic component on the perovskite support.

### BACKGROUND

Liquid-phase reactions can be industrially and environmentally advantageous, particularly due to the low temperatures, and optionally low pressures, involved. Examples of reactions that can advantageously be carried out in the liquid-phase are oxidation reactions, e.g. oxidation of alcohols to carboxylic acids, using molecular oxygen. In recent times the oxidation of glycerol has attracted significant attention due to the high functionality of glycerol and its availability from the trans-esterification of triglycerides as a by-product of the diesel manufacturing process.

Glycerol can be oxidised with heterogeneous catalysts to produce a range of oxygen added molecules with applications in polymers, building, cosmetics, food additives, and organic syntheses. Gold nanoparticles have been found to be active for the oxidation of glycerol in the presence of a base, such as sodium hydroxide. It has also been shown that a synergistic effect operates when gold is alloyed with another metal, such as palladium or platinum.

Many reactions comprise a plurality of competing reaction pathways. The oxidation of glycerol is an example of a reaction comprising competing reaction pathways.

The reaction mechanism for the oxidation of glycerol, shown in Scheme 1 of FIG. 1, contains multiple steps with a variety of different possible products. The initial step of the oxidation of glycerol is the formation of dihydroxyacetone, which is in equilibrium with glyceraldehyde. In the presence of a catalyst and base, under oxidising conditions, glyceraldehyde has been can be rapidly oxidised to glyceric acid, which can then be oxidised further. Glycerol can also be transformed under oxidative conditions to produce lactic acid. Lactic acid has many uses in the food industry and also to be polymerised to poly-lactic acid; a biodegradable material. The reaction pathway from glycerol to lactic acid proceeds via the dehydration of glyceraldehyde or dihydroxyacetone to form pyruvaldehyde, which then re-arranges into lactic acid.

The effect of various supports on the oxidation of glycerol in basic conditions has been studied. Carbon supports have been shown to be more active than titania and iron oxide supports (N. Dimitratos, J. A. Lopez-Sanchez, J. M. Anthonykutty, G. Brett, A. F. Carley, R. C. Tiruvalam, A. A. Herzing, C. J. Kiely, D. W. Knight and G. J. Hutchings, Physical Chemistry Chemical Physics, 2009, 11, 4952-4961). A study with Au/NiO and Au/NiO₁₋ₓ(TiO₂)ₓ showed very high activity, with the NiO support, but a poor selectivity to any particular product (A. Villa, G. M. Veith, D. Ferri, A. Weidenkaff, K. A. Perry, S. Campisi and L. Prati, Catal. Sci. Technol., 2013, 3, 394-399). Monometallic Au, Pd and Pt supported on activated carbon have been shown to be active for glycerol oxidation under base free conditions (A. Villa, G. M. Veith and L. Prati, Angewandte Chemie International Edition, 2010, 49, 4499-4502). Further studies have shown TiO₂, MgAl₂O₄ and H-mordenite supported gold catalysts have activity for glycerol oxidation in base free conditions (S. A. Kondrat, P. J. Miedziak, M. Douthwaite, G. L. Brett, T. E. Davies, D. J. Morgan, J. K. Edwards, D. W. Knight, C. J. Kiely, S. H. Taylor and G. J. Hutchings, ChemSusChem, 2014, 7, 1326-1334). Villa et al. studied the effect of acid and base properties of a support on the activity and selectivity of Au catalysts for the base free oxidation of glycerol (A. Villa, S. Campisi, K. M. H. Mohammed, N. Dimitratos, F. Vindigni, M. Manzoli, W. Jones, M. Bowker, G. J. Hutchings and L. Prati, Catal. Sci. Technol., 2015, 5, 1126-1132). The study found that basic supports resulted in high activity, but with the production of a large number of C1 and C2 scission products, while acid supports had lower activity but improved selectivity towards glyceraldehyde.

There is an ongoing need to develop catalysts having selectivity for particular reaction pathways to produce desired reaction products, in particular catalysts for liquid-phase reactions having selectivity for desired reaction products.

### SUMMARY OF THE INVENTION

The present inventors have found that perovskites, which are traditionally used in high temperature gas-phase reaction as catalysts, can be used as supports for catalytic components (as opposed to the perovskite being used as a catalyst itself) in lower temperature liquid-phase reactions and that the perovskite support allows the selectivity of the supported catalytic component to be tuned. The inventors have surprisingly found that the perovskite support which is not a catalytic component itself, but instead supports a catalytic component, can be employed to influence the selectivity of the catalytic component, for example, towards a desired reaction product of a selected reaction.

The present invention provides a supported catalyst, for example a supported catalyst for a liquid-phase reaction, the supported catalyst comprising: a perovskite support comprising an A-site species and a B-site species; and a catalytic component on a surface of the perovskite support. The B-site species may be selected to control the selectivity of the supported catalyst towards a desired reaction product.

The present invention also provides a method for forming a supported catalyst, for example a supported catalyst for a liquid-phase reaction, the method comprising: providing a perovskite support comprising an A-site species and a B-site species, wherein the B-site species is selected to control the selectivity of the supported catalyst towards a desired reaction product; and depositing a catalytic component on a surface of the perovskite support to form a supported catalyst.

The present invention provides a method for making a desired reaction product comprising: providing a supported catalyst having selectivity for the desired reaction product, the supported catalyst comprising: a perovskite support comprising an A-site species and a B-site species; and a catalytic component on a surface of the perovskite support, wherein the B-site species is selected to provide selectivity for the desired reaction product; and contacting reactants with the supported catalyst to provide the desired reaction product.

The present invention also provides a process for tuning the selectivity of a supported catalyst comprising a perovskite support comprising A-site and B-site species and a catalytic component deposited on the perovskite support, the process comprising varying the B-site species of the perovskite support to tune the selectivity of the supported catalyst.

The present invention also provides a process for identifying a supported catalyst having selectivity for a desired reaction product, the process comprising:
(a) selecting a reaction for producing the desired reaction product;
(b) selecting a catalytic component for catalysing the selected reaction;
(c) providing a plurality of supported catalysts, each supported catalyst comprising:
   a perovskite support comprising an A-site species and a B-site species; and
   the catalyst component on a surface of the perovskite support,
each of the supported catalysts having a different B-site species;
(d) carrying out the selected reaction using each of the supported catalysts provided in step (c); and
(e) determining the selectivity of each of the supported catalysts provided in step (c) for the desired reaction product.

In accordance with a first aspect of the present invention, there is provided a method for making a desired reaction product under liquid-phase conditions. The method may comprise:
providing a supported catalyst having selectivity for the desired reaction product, the supported catalyst comprising:
   a perovskite support comprising an A-site species and a B-site species; and
   metal or metal alloy catalytic particles deposited on a surface of the perovskite support,
   wherein the B-site species is selected to provide selectivity for the desired reaction product; and
contacting reactants with the supported catalyst to provide the desired reaction product.

In accordance with a second aspect of the present invention, there is provided a process for tuning the selectivity of a supported catalyst comprising a perovskite support comprising A-site and B-site species and metal or metal alloy catalytic particles deposited on the perovskite support, the process comprising varying the B-site species of the perovskite support to tune the selectivity of the supported catalyst.

In accordance with a third aspect of the present invention, there is provided a process for identifying a supported catalyst having selectivity for a desired reaction product. The process may comprise:
(a) selecting a reaction for producing the desired reaction product;
(b) selecting a metal or metal alloy for catalysing the selected reaction;
(c) providing a plurality of supported catalysts, each supported catalyst comprising:
   a perovskite support comprising an A-site species and a B-site species; and
   catalytic particles of the selected metal or metal alloy on a surface of the perovskite support,
each of the supported catalysts having a different B-site species;
(d) carrying out the selected reaction using each of the supported catalysts provided in step (c); and
(e) determining the selectivity of each of the supported catalysts provided in step (c) for the desired reaction product.

In accordance with a fourth aspect of the present invention, there is provided a method of forming a supported catalyst for a liquid-phase reaction. The method may comprise:
providing a perovskite support comprising an A-site species and a B-site species, wherein the B-site species is selected to control the selectivity of the supported catalyst towards a desired reaction product;
depositing metal or metal alloy catalytic particles on a surface of the perovskite support to form a supported catalyst; and
exposing the supported catalyst to a temperature not greater than about 350° C. such that the metal or metal alloy catalytic particles remain on the surface of the perovskite support.

In accordance with a fifth aspect of the present invention, there is provided a perovskite supported catalyst for a liquid-phase reaction. The supported catalyst may comprise:
a perovskite support comprising an A-site species and a B-site species; and
metal or metal alloy catalytic particles on a surface of the perovskite support.

In accordance with a sixth aspect of the present invention, there is provided the use of the supported catalyst described herein in a selective reaction, e.g. a liquid-phase reaction, to produce a desired reaction product.

### BRIEF DESCRIPTION OF THE DRAWINGS

FIG. 1 is a technical diagram of a Separex SAS apparatus.

FIG. 2 shows the reaction mechanism for the oxidation of glycerol (Scheme 1).

FIG. 3 is a graph showing the thermogravimetric analysis of SAS precipitated materials.

FIG. 4 shows powder X-ray diffraction patterns of SAS La:B precipitates as described in the Examples.

FIG. 5a shows a representative transmission electron micrograph (TEM) of a supported catalyst where the perovskite support is LaCrO₃ and the catalytic component is AuPt.

FIG. 5b shows a representative TEM of a supported catalyst where the perovskite support is LaMnO₃ and the catalytic component is AuPt.

FIG. 5c shows a representative TEM of a supported catalyst where the perovskite support is LaFeO₃ and the catalytic component is AuPt.

FIG. 5d shows a representative TEM of a supported catalyst where the perovskite support is LaCoO₃ and the catalytic component is AuPt.

FIG. 5e shows a representative TEM of a supported catalyst where the perovskite support is LaNiO₃ and the catalytic component is AuPt.

FIG. 6a shows a particle size distribution histograms of AuPt supported on a SAS prepared LaCrO₃ perovskite support.

FIG. 6b shows a particle size distribution histograms of AuPt supported on a SAS prepared LaMnO₃ perovskite support.

FIG. 6c shows a particle size distribution histograms of AuPt supported on a SAS prepared LaFeO₃ perovskite support.

FIG. 6d shows a particle size distribution histograms of AuPt supported on a SAS prepared LaCoO₃ perovskite support.

FIG. 6e shows a particle size distribution histograms of AuPt supported on a SAS prepared LaNiO₃ perovskite support.

FIG. 7 shows a graph showing the conversion of glycerol with AuPt/LaBO₃ catalysts, where the B sites of the supports are; Cr (●); Mn (▪); Fe (▴); Co (□); Ni (◯).

FIG. 8 shows a conversion-selectivity plots for glyceric acid, tartronic acid, C—C scission and lactic acid selectivity from the glycerol oxidation reaction using AuPt/LaBO₃ supported catalysts, where the B sites of the supports are; Cr (●); Mn (▪); Fe (▴); Co (□); Ni (◯).

FIG. 9 shows selectivity profiles of AuPt/LaBO₃ supported catalysts compared to reported oxygen adsorption values for the relevant perovskite phases.

FIG. 10 provides graphs showing time on line conversion and selectivity (left) and time on line molar concentration (right) plots for extended glycerol oxidation reaction time using a AuPt/LaMnO₃ supported catalyst.

### DETAILED DESCRIPTION OF THE INVENTION

The term “perovskite” as used herein is used to refer to a perovskite-type oxide, for example a perovskite-type oxide having the general formula ABO₃, where A and B are cations and cation A is larger than cation B. The general formula ABO₃ described herein encompasses the formula ABₓO₃, where x ranges from about 0.9 to about 1.1, for example about 0.95 to about 1.05, or about 0.99 to about 1.01. In certain embodiments x is about 1.

The term “perovskite support” refers to a perovskite for supporting a catalytic component, for example, the perovskite support may not be a catalytic component itself but supports a catalytic component. The perovskite support may be provided to support a catalytic component for catalysing a reaction to obtain a desired reaction product.

The perovskite support may be inactive in the absence of the catalytic component supported on the perovskite support, i.e. if the perovskite support alone (i.e. in the absence of a catalytic component) is attempted to be used as a catalyst for the selected reaction, the activity is substantially the same (for example the difference in activity may be about 10% or less for the selected reaction in the presence of the perovskite support alone and the selected reaction with no perovskite support) as the activity in the absence of the perovskite support (i.e. the absence of the perovskite support or any catalytic component). For example, ‘substantially the same activity’ may mean that there is a difference of less than about 20%, for example about 10% or less, between the conversion of the selected reaction carried out in the presence of the perovskite support alone (the perovskite support with no catalytic component) and the selected reaction carried out with no perovskite support (i.e. no perovskite support or catalytic component). The perovskite support may provide no selectivity for a desired reaction product of a selected reaction in the absence of a catalytic component, i.e. if the perovskite support alone (i.e. the perovskite support with no catalytic component) is attempted to be used as a catalyst for the selected reaction, the selectivity for the desired reaction product is substantially the same, or the same, as the selectivity for the desired reaction product in the absence of the perovskite support (i.e. in the absence of a perovskite support or catalytic component). A perovskite, perovskite-type oxide or modified perovskite described herein may have a cubic, orthorhombic or rhombohedral crystal structure. A perovskite, perovskite-type oxide or modified perovskite described herein may be identified using powder X-ray diffraction (XRD) and comparing the resulting XRD pattern with a database of well-defined crystalline materials (e.g. the ICDD (International Centre for Diffraction Data) database).

The perovskite support comprises an A-site species and a B-site species. The term “A-site species” is used herein to describe a species occupying A cation sites in a perovskite-type oxide, for example a perovskite type oxide having the general formula ABO₃. The term “B-site species” is used herein to describe a species occupying B cation sites in a perovskite-type oxide, for example a perovskite type oxide having the general formula ABO₃. The A-site species may be any metal cation suitable for occupying the A-site of a perovskite. In certain embodiments, the A-site species may be selected from alkaline earth metal cations, (i.e. metal cations selected from Group 2 of the Periodic Table), lanthanide cations and combinations thereof. The B-site species may be any metal cation suitable for occupying the B-site of a perovskite. In certain embodiments, the B-site species may be selected from transition metal cations (i.e. a metal cation selected from any of Groups 4 to 12 of the Periodic) or combinations thereof. In certain embodiments, the A-site species may comprise more than one species. In certain embodiments, the B-site species may comprise more than one species.

The perovskite support may be referred to herein as a modified perovskite support. The term “modified perovskite” may be used to describe a perovskite in which the B-site species has been selected, for example to control the selectivity of the supported catalyst, a perovskite having a BET surface area of about 15 m²/g or greater, and/or a perovskite having a crystallite size of less than about 50 nm.

The BET surface area of a perovskite support may be measured using Quadrasorb equipment and a BET method in which a 5 point isotherm of nitrogen adsorption at −196° C. is taken to provide a straight line of which the gradient provides the surface area of the perovskite support. In certain embodiments, the BET surface area of the perovskite support may be determined according to ASTM D3663.

If a standard test is mentioned herein, unless otherwise stated, the version of the test to be referred to is the most recent at the time of filing this patent application.

The perovskite support crystallite size, or crystallite size of the supported catalyst may be determined as the average domain length (maximum dimension) of the perovskite lattice plane providing the principle reflection, for example the (121) lattice plane of the perovskite. In order to determine the crystallite size using XRD, the principle reflection is determined from a powder XRD pattern for the particular perovskite and the Scherrer equation is used to determine the crystallite size from the full width half maximum of the peak and peak position of the principle reflection using a 0.9 shape factor (see for example Spectroscopy in Catalysis, 3^{rd} edition, J. W. Niemantsverdriet, chapter 6.2, page 151, Wiley-VCH).

Described herein is a process for tuning the selectivity of a supported catalyst comprising a perovskite support. The term “tuning” is used herein to describe modification of the composition of a perovskite support without changing the structure of the perovskite support to provide a supported catalyst having selectivity for a particular product or products of a selected reaction. The present inventors have found that varying the B-site species of a perovskite support allows the selectivity of a supported catalyst comprising the perovskite support to be tuned to favour the production of a desired product or products of a selected reaction over competing and less-desired reaction products.

The term “catalytic component” used herein refers to a component for catalysing a selected reaction that is present on a surface of a perovskite support in the supported catalyst. The catalytic component may comprise, consist essentially of, or consist of catalytic particles, for example catalytic nanoparticles. Catalytic nanoparticles may have an average particles size of less than about 20 nm, for example less than about 10 nm, for example less than about 5 nm. In certain embodiments the catalytic nanoparticles may have an average particles size between about 0.1 and about 10 nm. The catalytic component may comprise a metal, for example a metal or a metal alloy, for example metal catalytic particles. The metal particles may be monometallic or may comprise more than one metal such as a metal alloy. The metal alloy may be bimetallic.

The catalytic component may consist essentially of or consist of a metal or metal alloy, for example metal or metal alloy catalytic particles. In certain embodiments, the metal or metal alloy catalytic particles may be metal or metal alloy nanoparticles, for example metal or metal alloy particles having an average particle size of less than about 20 nm, for example less than about 10 nm, for example less than about 5 nm. In certain embodiments, the metal or metal alloy catalytic particles may be metal or metal alloy nanoparticles, for example metal or metal alloy particles having an average particle size between about 0.1 and about 10 nm, for example between about 0.25 nm and about 10 nm, for example between about 0.25 nm and about 5 nm.

The average particle size of the metal or metal alloy nanoparticles may be determined using transmission electron microscopy (TEM) or scanning transmission electron microscopy (STEM). For example, TEM or STEM may be used to measure the particle size (largest dimension of the particle) of each of a number of particles, for example each of about 500 nanoparticles, on the surface of a perovskite support and the average particle size of the measured nanoparticles calculated.

The metal or metal alloy nanoparticles may have a particle size distribution of between about 0.1 and about 20 nm, for example between about 0.1 and about 10 nm, for example between about 0.1 and about 5 nm, or between about 0.25 to 5 nm. The particle size distribution may be determined using a transmission electron microscopy (TEM) or a scanning transmission electron microscopy (STEM). For example, TEM or STEM may be used to measure the particle size (largest dimension) of each of a number of nanoparticles, for example about 100 nanoparticles, on the surface of a perovskite support and the upper and lower particle sizes determined. In certain embodiments, at least about 60%, in certain embodiments at least about 70%, in certain embodiments at least about 80%, in certain embodiments at least about 90%, of the metal of metal alloy nanoparticles may have a particle size in the range about 0.1 to about 10 nm, in for example about 0.1 to about 5 nm, or about 0.25 to about 5 nm.

Metal catalytic particles may comprise any metal or combination of metals suitable for catalysing a selected reaction. In certain embodiments the metal catalytic particles may be metal particles containing a single metal, i.e. monometallic metal catalytic particles, or metal particles containing more than one metal, e.g. the metal particles may be bimetallic particles, such as metal alloy catalytic particles. In certain embodiments, the metal catalytic particles, for example the metal or metal alloy catalytic particles, may comprise a transition metal or a combination of transition metals. In certain embodiments, the metal catalytic particles, for example the metal or metal alloy catalytic particles, may comprise gold, platinum, palladium, ruthenium, rhodium, silver, copper or combinations thereof, in certain embodiments gold and platinum.

The supported catalyst described herein comprises a perovskite support and a catalytic component on a surface of the perovskite support. As used herein, the phrase “on a surface of the perovskite support” refers to the catalytic component, e.g. metal or metal alloy catalytic particles, being present on the support as opposed to being incorporated into the perovskite structure.

In certain embodiments, the supported catalyst may comprise gold, platinum, palladium, ruthenium, rhodium, silver, copper atoms or combinations thereof within the perovskite support.

The term “liquid-phase reaction” as used herein, refers to a reaction in which at least one reactant is in the liquid-phase, e.g. a reaction carried out at a temperature and/or pressure such that at least one of the reactants is liquid or in solution in a liquid. For example, in the liquid-phase oxidation of glycerol in which glycerol, oxygen and a supported catalyst described herein are contacted, glycerol is liquid glycerol. A reactant in the liquid phase may also refer to a reactant in a liquid solution, for example an aqueous solution.

The present inventors have surprisingly found that it is possible to tune the selectivity of a supported catalyst comprising a perovskite support by varying the B-site species of the perovskite support. For example, without varying the catalytic component, such as the metal or metal alloy catalytic particles, on a surface of the perovskite support.

Supported Catalyst and Supported Catalyst Preparation

The supported catalyst, for example a supported catalyst for a liquid-phase reaction, may comprise:
a perovskite support comprising an A-site species and a B-site species; and
a catalytic component on a surface of the perovskite support.

The B-site species may be selected to control the selectivity of the supported catalyst towards a desired reaction product.

The catalytic component may be metal catalytic particles, e.g. metal or metal alloy catalytic particles. The catalytic component, e.g. metal (e.g. metal or metal alloy) catalytic component may be as described above.

In certain embodiments, the metal catalytic particles, e.g. metal or metal alloy catalytic particles, comprise gold, platinum, palladium, ruthenium, rhodium, silver, copper or combinations thereof, for example gold, platinum, palladium or combinations thereof. In certain embodiments, the catalytic particles comprise gold and platinum, for example a gold and platinum alloy.

In certain embodiments, the metal catalytic particles are bimetallic metal particles, e.g. metal alloy particles, comprising a first metal and a second metal. In certain embodiments the first metal and the second metal are different and are both selected from transition metals. In certain embodiments the first and second metals are different and are selected from gold, platinum, palladium, ruthenium, rhodium, silver and copper.

In certain embodiments the catalytic particles comprise a metal alloy comprising a first metal and a second metal in bulk ratio of first metal to second metal of about 0.3 to about 3, for example about 0.5 to about 2, or about 0.8 to about 1.2, or about 0.9 to about 1.1. The bulk ratio of the first metal to the second metal may be determined by microwave plasma atomic emission spectroscopy (MP-AES), for example, using an Agilent 4100 instrument. In certain embodiments, the surface ratio of first metal to second metal from X-ray photoelectron spectroscopy (XPS) is about 0.5 to about 1.5. The surface ratio of first metal to second metal may be deterred using a Kratos Axis Ultra DLD system with a monochromatic Al K_{α} X-ray source operating at 120 W.

In certain embodiments the catalytic particles are bimetallic catalytic particles, e.g. metal alloy catalytic particles, comprising gold as the first metal and platinum as the second metal.

In certain embodiments, the supported catalyst comprises at least about 0.1 wt. % of a catalytic component, for example metal or metal alloy catalytic particles (e.g. nanoparticles), by total weight of supported catalyst, for example about 0.5 wt. % or greater, for example about 0.75 wt. % or greater, for example about 1 wt. %, e.g. about 1.0 wt. % or greater, for example about 2 wt. %, e.g. about 2.0 wt. % or greater, for example about 3 wt. %, e.g. 3.0 wt. % or greater, for example about 4 wt. %, e.g. 4.0 wt. % or greater, in certain embodiments about 5 wt. %, e.g. about 5.0 wt. % or greater by totally weight of supported catalyst. In certain embodiments, the supported catalyst comprises at least about 0.1 wt. % of catalytic particles, for example metal or metal alloy catalytic particles (e.g. nanoparticles), by total weight of supported catalyst on a surface of the perovskite support, in certain embodiments at least 0.5 wt. %, in certain embodiments at least 0.75 wt. %, for example at least about 1 wt. %, e.g. about 1.0 wt. %, for example at least about 2 wt. %, e.g. about 2.0 wt. %, or at least about 3 wt. %, e.g. about 3.0 wt. %, or at least about 4 wt. %, e.g. about 4.0 wt. %, or at least about 5 wt. %, e.g. about 5.0 wt. %, of catalytic particles, for example metal or metal alloy catalytic particles (e.g. nanoparticles), by total weight of supported catalyst on a surface of the perovskite support.

In certain embodiments, the supported catalyst comprises between about 0.1 wt. % and about 10 wt. % of a catalytic component, for example metal or metal alloy catalytic particles (e.g. nanoparticles), by total weight of supported catalyst, in certain embodiments between about 1 wt. % and about 10 wt. %, for example between 1 wt. % and 5 wt. % by total weight of supported catalyst.

In certain embodiments, the perovskite support of the supported catalyst has a BET surface area of greater than about 15 m²/g, for example at least about 20 m²/g, at least about 22 m²/g, at least about 25 m²/g, or at least about 30 m²/g. In certain embodiments, the perovskite support of the supported catalyst has a BET surface area in the range of about 20 m²/g to about 80 m²/g.

The A-site species may be any metal cation suitable for occupying the A-site of a perovskite. The A-site species may be any metal cation that has a larger ionic radii than the ionic radii of the B-site species. The A-site species may be any metal cation having a +2, +3, or +4 oxidation state. The A-site species may be selected from the group comprising or consisting of alkali metal cations, alkaline earth metal cations, lanthanide cations or combinations thereof. The A-site species may be selected from the group comprising or consisting of alkaline earth metal cations and lanthanide cations. In certain embodiments, the A-site species is a lanthanum cation.

The B-site species may be any metal cation suitable for occupying the B-site of a perovskite. The B-site species may be any metal cation that has a smaller ionic radii than the ionic radii of the A-site species The B-site species may be selected from the group comprising or consisting of transition metal cations. For example, the B-site species may be selected such that the B-site species has an oxidation state which is stable with respect to the oxidation state of the A-site species. In certain embodiments, the B-site species is selected from chromium, manganese, iron, cobalt and nickel cations.

In certain embodiments, the A-site species is a lanthanum cation and the B-site species is selected such that the B-site species has an oxidation state which is stable with respect to the oxidation state of the lanthanum cation, for example, the B-site species may be selected chromium, manganese, iron, cobalt and nickel cations.

In certain embodiments, the supported catalyst has a crystallite size of less than about 100 nm, for example less than about 50 nm, or less than about 25 nm. In certain embodiments, the crystallite size of the supported catalyst is in the range of about 1 to about 50 nm, for example about 5 to about 50 nm, or about 2 to about 25 nm. The crystallite size may be measured using XRD as described above.

In certain embodiments, the supported catalyst has an average particle size of less than 100 nm, in certain embodiments less than about 50 nm. In certain embodiments, the supported catalyst has an average particle size in the range of about 1 to about 100 nm, for example about 1 to about 50 nm, about 2 to about 50 nm, about 5 to about 50 nm, or about 1 to about 25 nm. The average particle size of the supported catalyst may be determined using TEM, for example by measuring the particle size (largest dimension of the particle) of a number of particles, for example about 500 particles, and calculating the average.

Also described herein is a method of forming a supported catalyst described herein.

The method of forming a supported catalyst, e.g. a supported catalyst for a liquid-phase reaction, may comprise:
providing a perovskite support comprising an A-site species and a B-site species, wherein the B-site species is selected to control the selectivity of the supported catalyst towards a desired reaction product; and
depositing a catalytic component on a surface of the perovskite support.

In certain embodiments, the catalytic component comprises, consists essentially of, or consists of metal or metal alloy catalytic particles, e.g. metal or metal alloy nanoparticles.

In certain embodiments the method comprises exposing the supported catalyst to a temperature not greater than about 350° C., for example not greater than about 320° C., not greater than about 300° C., not greater than about 250° C., not greater than about 200° C., or not greater than about 175° C., such that metal or metal alloy particles of the catalytic component remain on the surface of the perovskite support.

In certain embodiments the maximum temperature to which the supported catalyst may be exposed may be determined by the Hüttig temperature of the catalytic component, for example the Hüttig temperature of a metal of the metal or metal alloy catalytic particles.

In certain embodiments the method comprises exposing the supported catalyst to a temperature not greater than the Hüttig temperature of the catalytic component.

The Hüttig temperature of the catalytic component is a temperature above which ions within the bulk of the catalytic component, for example metal ions of the metal or metal alloy catalytic particles, are sufficiently mobile to begin to agglomerate and sinter. The Hüttig temperature of the catalytic component, e.g. a metal of the metal or metal alloy catalytic particles, may be affected by the interaction between the catalytic component, a metal of the metal or metal alloy catalytic particles, and the surface of the perovskite. For example, the Hüttig temperature can be taken to be a temperature which is one third of the temperature of the melting point of the catalytic component, e.g. one third of the melting point of a metal of metal catalytic particles, or part of the catalytic component (e.g. the Hüttig temperature of the catalytic component with the lowest melting point, e.g. the Hüttig temperature of the metal of the metal alloy having the lowest melting temperature). In certain embodiments the method comprises exposing the supported catalyst to a temperature not greater than a temperature which is one third of the melting point of the metal or a metal of the metal alloy of the catalytic component.

The step of providing a perovskite support may comprise identifying a perovskite support suitable for supporting metal or metal alloy catalytic particles to provide a supported catalyst having selectivity for the desired reaction product. The process of identifying a suitable perovskite support may comprise varying the B-site species to tune the selectivity of the supported catalyst comprising a particular catalytic component for a desired reaction product.

Selecting of the B-site species to control the selectivity of the supported catalyst towards a desired reaction product may comprise the process for identifying a supported catalyst having selectivity for a desired reaction described below.

In certain embodiments, the perovskite support has a BET surface area of greater than about 15 m²/g, for example greater than about 20 m²/g, greater than about 22 m²/g, greater than about 25 m²/g, or greater than about 30 m²/g. The present inventors have found that perovskite supports having a high surface area, e.g., such as greater than about 15 m²/g, can be provided to support a catalytic component such that the amount of the catalytic component is sufficient to catalyst a reaction. For example, the supported catalyst may comprises at least about 0.1 wt. %, for example at least about 0.5 wt. %, at least about. 0.75 wt. % or about 1 wt. %, of a catalytic component on a surface of a perovskite support by total weight of the supported catalyst.

Perovskite supports having a BET surface area of greater than about 15 m²/g, or greater than about 20 m²/g, may be formed using methods known by the skilled person. For example, high area perovskites supports may be provided using a supercritical anti-solvent precipitation (SAS) method, for example as described in the Examples section that follows, or a flame pyrolysis method. The perovskite supports may also be provided using co-precipitation, citrate preparation, or hard templating methods known to the skilled person.

The perovskite support prepared according to one of the above-mentioned methods may be calcined before a catalytic component is deposited on the surface of the perovskite support. Calcining of the perovskite support may be carried out at a temperature of greater than about 400° C., for example greater than about 500° C., greater than about 600° C., or greater than about 700° C. In certain embodiments calcining is carried out at a temperature below about 900° C., for example below about 800° C., or below about 750° C. In certain embodiments calcining is carried out at a temperature in the range about 700 to about 800° C. In certain embodiments at a temperature of about 750° C. The present inventors have found that such calcination temperatures allow a balance between high phase purity and high surface area to be achieved.

The catalytic component, e.g. the metal or metal alloy catalytic particles, may be deposited on a surface of the perovskite support by any method known to the skilled person. For example, the metal or metal alloy catalytic particles may be deposited on a surface of the perovskite support by impregnating the perovskite support with an aqueous solution comprising a component containing the metal or metals of the metal alloy to be deposited on the perovskite support. For example, the perovskite support may be impregnated in an aqueous solution containing ions of the meal or metals of the metal alloy to be deposited on the perovskite support. A reducing agent, such as NaBH₄, may be added to the aqueous solution containing the perovskite support such that metal or metal alloy catalytic particles are deposited on a surface of the perovskite support. The perovskite support on which the metal or metal alloy catalytic particles have been deposited may then be removed from the aqueous solution. The supported catalyst comprising the perovskite support and metal or metal alloy particles on a surface of the perovskite support may then be dried, for example at a temperature of less than about 150° C., e.g. about 120° C., to remove water.

In certain embodiments, in the method of forming a supported catalyst as described herein, the supported catalyst (comprising the perovskite support and the metal or metal alloy catalytic particles deposited on a surface of the perovskite support) should not be exposed to temperatures greater than the Hüttig temperature or the metal or a metal of the metal alloy. In certain embodiments, in the method of forming a supported catalyst as described herein, the supported catalyst (comprising the perovskite support and the metal or metal alloy catalytic particles deposited on a surface of the perovskite support) should not be exposed to temperatures greater than about 350° C., for example not greater than about 320° C., not greater than about 300° C., not greater than about 250° C., not greater than about 200° C., or not greater than about 175° C. In certain embodiments the supported catalyst should not be exposed to temperatures greater than 150° C. By avoiding exposing the supported catalyst to high temperatures, e.g. temperatures in excess of the Hüttig temperature of the metal or a metal of the metal or metal alloy catalytic particles, for example temperatures greater than about 350° C., sintering of the catalytic particles can be avoided. It is also thought that by avoiding exposing the supported catalyst to such high temperatures, diffusion or agglomeration of the metal or metal alloy catalytic particles can be avoided. For example, avoiding exposing the supported catalysts to high temperatures, e.g. temperatures about the Hüttig temperature of a metal of the metal or metal alloy catalytic particles, may prevent incorporation of components of the catalytic particles, for example metal of the metal or metal alloy catalytic particles, into the perovskite support. Thus in certain embodiments the supported catalyst is not exposed to temperatures above the Hüttig temperature of the catalytic component.

The supported catalyst may therefore be suitable for a liquid-phase reaction at which temperatures are below about 350° C., in certain embodiments less than about 320° C., for example less than about 300° C., less than about 250° C., less than about 200° C., or less than about 175° C. or less than about 150° C. In certain embodiments, the supported catalyst may not be suitable for gas phase reactions which may occur at higher temperatures, e.g. temperatures greater than about 350° C., or for example greater than about 300° C., or greater than about 250° C., or greater than about 200° C., or greater than about 175° C., or greater than about 150° C.

Tuning of Supported Catalyst and Identification of a Supported Catalyst Having Selectivity for a Desired Reaction Product

Optional and preferred features of the supported catalyst, including the perovskite support and the catalytic component, discussed above also apply to these embodiments.

Described herein is a process for tuning the selectivity of a supported catalyst comprising a perovskite support comprising A-site and B-site species and catalytic component, such as metal or metal alloy catalytic particles, deposited on the perovskite support, the process comprising varying the B-site species of the perovskite support to tune the selectivity of the supported catalyst.

The process for tuning may comprise varying the B-site species of the perovskite support while the A-site species of the perovskite support and the catalytic component, e.g. the metal or metal catalytic particles, on the perovskite support are unchanged.

In certain embodiments, the supported catalyst comprises a perovskite support and a catalytic component on a surface of the perovskite support.

The process for tuning the selectivity of the supported catalyst may comprise selecting a reaction for producing a desired reaction product and selecting a catalytic component, e.g. a metal or metal alloy such as metal or metal alloy catalytic particles, for catalysing the selected reaction to produce the desired reaction product.

The process for tuning the selectivity of the supported catalyst may comprise screening a number of different supported catalysts, each comprising a different perovskite support, for selectivity towards the desired reaction product in the selected reaction. Each different supported catalyst screened may comprise the same catalytic component on a different perovskite support, each different perovskite support comprising the same A-site species but a different B-site species. Each different supported catalyst may then be used to catalyse the selected reaction and selectivity of each of the supported catalysts for the desired reaction product determined. In certain embodiments, the reaction conditions for each of the reactions catalysed by each of the different supported catalysts are the same. In certain embodiments, once a supported catalyst or supported catalysts having appropriate selectivity, e.g. the highest selectivity, for the desired reaction product is selected the reaction conditions may then be modified in order to further improve the selectivity of the supported catalyst(s) for the desired reaction product.

The features described below relating to the process for identifying a supported catalyst having selectivity for a desired reaction are also applicable to the process for tuning the selectivity of the supported catalyst.

Also described herein is a process for identifying a supported catalyst having selectivity for a desired reaction product, the process comprising:
(a) selecting a reaction for producing the desired reaction product;
(b) selecting a metal or metal alloy for catalysing the selected reaction;
(c) providing a plurality of supported catalysts, each supported catalyst comprising:
   a perovskite support comprising an A-site species and a B-site species; and
   catalytic particles of the selected metal or metal alloy on a surface of the perovskite support,
each of the supported catalysts having a different B-site species;
(d) carrying out the selected reaction using each of the supported catalysts provided in step (c); and
(e) determining the selectivity of each of the supported catalysts provided in step (c) for the desired reaction product.

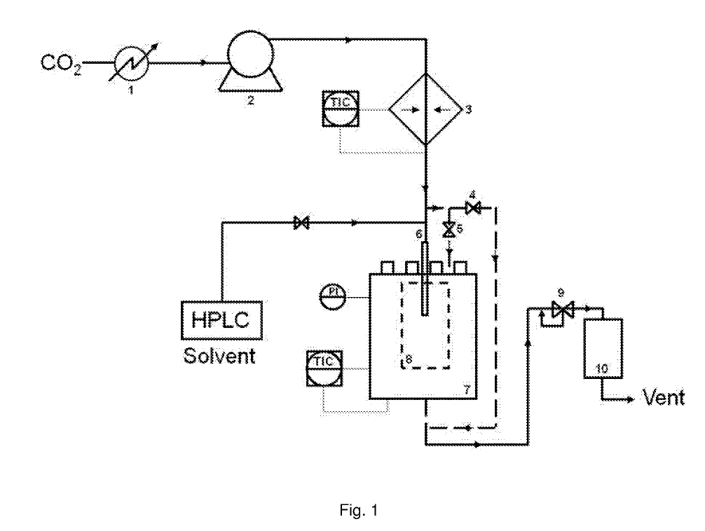

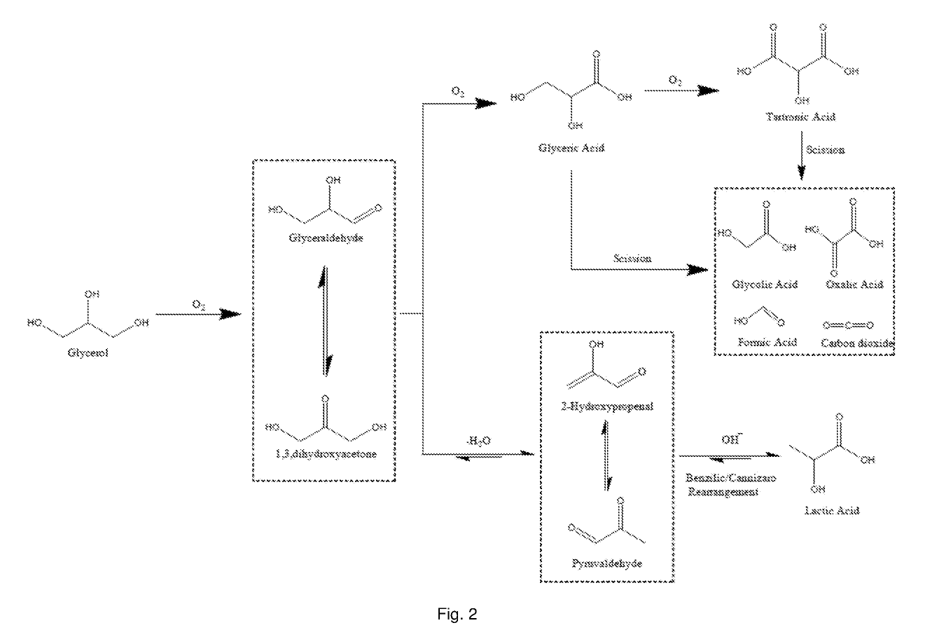

Selecting a reaction for producing the desired reaction product may comprise selecting a reaction comprising competing reaction pathways, wherein one of the competing reaction pathways leads to the desired reaction product and another of the competing reaction pathways leads to a competing product. For example, the desired reaction product may be selected as tartronic acid in the oxidation of glycerol reaction which comprises competing oxidation, scission and dehydration pathways as outlined in scheme 1 shown in FIG. 1.

Selecting a metal or metal alloy for catalysing the selected reaction may comprise selecting a metal or metal alloy that may be used unsupported, or supported on a different support (such as a carbon support or a titanium support for example), to catalyse the selected reaction. For example, gold nanoparticles or metal alloy nanoparticles comprising gold and platinum are known to catalyse the reaction of glycerol oxidation. Therefore, if the selected reaction is the oxidation of glycerol, gold metal or Au/Pt metal alloy may be selected as the metal/metal alloy for catalysing the selected reaction.

Providing a plurality of supported catalyst may comprise forming a number of supported catalysts according to the method described in which each of the supported catalysts comprise the same A-site species and catalytic component but a different B-site species.

The selected reaction may be carried out using each of the supported catalysts. In certain embodiments, the reaction conditions for each reaction using a different supported catalyst are the same. In certain embodiments the reaction conditions are selected such that the reaction produces the broadest range of products in the absence of a supported catalyst. In certain embodiments, the reaction conditions may be selected to produce a desired reaction product in the absence of a supported catalyst. For example, in the example of the reaction of the oxidation of glycerol, the reaction conditions may be selected to provide lactic acid.

The selectivity of each of the supported catalysts for the desired reaction product, in certain embodiments for each of the reaction products, under pre-determined reaction conditions may then be determined. In certain embodiments, the selectivity of each of the supported catalysts for the desired reaction product or each of the reaction products may then be determined under different reaction conditions.

The selectivity of each of the supported catalysts may be determined by determining the amount of desired reaction product produced compared to the total amount of product formed from the converted reactants.

Production of a Desired Reaction Product

Embodiments and features described in relation to the supported catalyst, including the perovskite support and the catalytic component, the process of tuning the selectivity of the supported catalyst and the process for identifying a supported catalyst having selectivity for a desired reaction product discussed above also apply to the process for producing a desired reaction product and the use of a supported catalyst to product a desired reaction product.

Also described herein is a method for making a desired reaction product under liquid-phase conditions, the method comprising:
providing a supported catalyst having selectivity for the desired reaction product, the supported catalyst comprising:
   a perovskite support comprising an A-site species and a B-site species; and
   metal or metal alloy catalytic particles on a surface of the perovskite support,
   wherein the B-site species is selected to provide selectivity for the desired reaction product; and
contacting reactants with the supported catalyst to provide the desired reaction product.

In certain embodiments, the step of contacting reactants with the supported catalyst is carried out a temperature of less than about 350° C., for example less than about 320° C., for example less than about 300° C., for example less than about 250° C., for example less than about 200° C. or less than about 150° C., to ensure that the metal or metal alloy catalytic particles remain on a surface of the perovskite support during the reaction.

In the example in which the reactants comprise glycerol and oxygen, the B-site species may be selected such that the supported catalyst has selectivity for the oxidation of glycerol. In certain embodiments, the desired reaction product is glyceric acid, tartronic acid or lactic acid and the B-site species is selected such that the supported catalyst has selectivity for glyceric acid, tartronic acid or lactic acid.

Each of the features of each of the embodiments described above may be combined with the features of each of the other embodiments described above.

Embodiments of the present invention will now be described by way of illustration only, with reference to the following examples.

### EXAMPLES

Perovskite Support Preparation

A range of LaBO₃ (B denotes Cr, Mn, Fe, Co or Ni) perovskites were prepared using the supercritical anti-solvent precipitation (SAS) method. A brief summary of the preparation method is given below, with a more detailed experimental method reported by Marin et al. (R. P. Marin, S. A. Kondrat, R. K. Pinnell, T. E. Davies, S. Golunski, J. K. Bartley, G. J. Hutchings and S. H. Taylor, Applied Catalysis B: Environmental, 2013, 140-141, 671-679). Lanthanum (III) acetylacetonate hydrate (4 mg ml⁻¹) and one of the B element acetate salts (concentration varied to give La:B molar ratios shown in Table 1) (Sigma Aldrich ≥99% Puriss), were dissolved in methanol (reagent grade, Fischer Scientific).

Supercritical anti-solvent (SAS) experiments were performed using apparatus manufactured by Separex. A technical diagram of the SAS apparatus is shown in FIG. 1 in which the reference numeral denote the following: (1) Chiller; (2) liquid pump; (3) heat exchanger; (4) and (5) by-pass valves, (6) co-axial nozzle for CO2 and metal salt solution delivery; (7) precipitation vessel; (8) sample recovery vessel; (9) back pressure regulator and (10) separation vessel. CO₂ (BOC) was pumped through the system (held at 130 bar, 40° C.) via the outer part of a co-axial nozzle at a rate of 12 kg h⁻¹. The metal salt solution was co-currently pumped through the inner nozzle, using an Agilent HPLC pump at a rate of 4 ml min⁻¹. The resulting precipitate was recovered on a stainless steel frit, while the CO₂-solvent mixture passed down stream, where the pressure was decreased to separate the solvent and CO₂. The precipitation vessel has an internal volume of 1 L. Precipitation was carried out for 120 min followed by a purge of the system with CO₂ for 30 min under 130 bar and 40° C. The system was then depressurised and the dry powder collected. The SAS precipitates were then calcined at 750° C. (with a ramp rate of 2° C. min⁻¹) for 4 h to produce the perovskite materials.

Addition of Catalytic Component to Perovskite Supports

As an example, the reaction investigated was the oxidation of glycerol. As a Au/Pt alloy is known to be useful in catalysing the oxidation of glycerol, Au/Pt nanoparticles were deposited on each of the perovskite supports.

Aqueous solutions of HAuCl₄ (Johnson Matthey) and H₂PtCl₆ (Johnson Matthey) were prepared at a such that the molar ratio of Au:Pt was 1:1. Polyvinyl alcohol (PVA, 1 wt % aqueous solution, Aldrich, MW=10 kDa) was freshly prepared and used as the stabilizer. NaBH₄ (Sigma Aldrich, 0.1 M aqueous solution) was also freshly prepared and used as the reducing agent. To an aqueous mixture of HAuCl₄ and H₂PtCl₆ of the desired concentration (1:1 metal weight ratio, 1 wt % total metal in final catalyst) the PVA solution was added (PVA/(Au+Pt) (wt/wt)=0.65) with vigorous stirring for 2 min. NaBH₄ was then added rapidly such that the NaBH₄:total metal ratio (mol/mol) was 7.5. After 1 h of stirring the mixture was filtered, washed with distilled water and dried at 120° C. for 16 h. The resulting supported catalysts were not calcined.

Supported Catalyst Characterisation

The ratio of the A-site species:B-site species of the SAS precipitated perovskites were determined by microwave plasma atomic emission spectroscopy (MP-AES) using an Agilent 4100 instrument (results shown in Table 1 above). The precipitates were dissolved to form 10, 30 and 50 ppm solutions and the La content was determined using the 394,910 and 398,852 nm emission lines. The emission lines used for the B-site species were as follows: 357,688 and 425,433 nm for Cr, 403,076, 403,307 nm for Mn, 259,940, 371,993 nm for Fe, 340,512, 345,351 nm for Co and 341,476, 352,454 nm for Ni. The Au content was determined using the 242,795, 267,595 nm emission lines and Pt content was determined from the 265,945, 270,240 nm emission lines (results shown in Table 2 below).

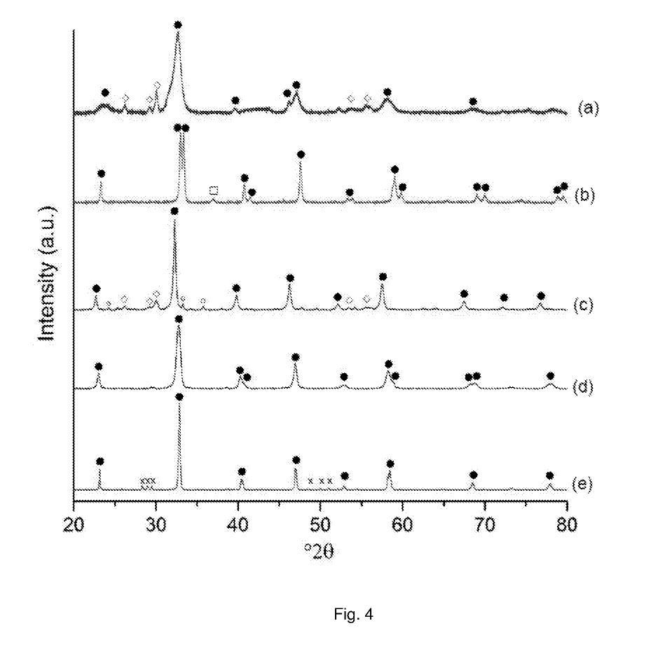

Powder X-ray diffraction (XRD) was used to determine the phase purity of the prepared perovskites. X-ray diffraction data were collected using the La:B precipitates formed using the SAS method above after calcining at 750° C. on a Panalytical X'Pert diffractometer, with Cu K_{α1} radiation, operating at 40 kV and 40 mA (XRD patterns shown in FIG. 4 in which lines (a)-(e) show the results for materials in which the B-site species was (a) Ni, (b) Co, (c) Fe, (d) Mn and (e) Cr. the phases present are identified as follows: ● perovskite phases (for simplicity rhombohedral, orthorhombic and cubic phases are not differentiated); ◯ Fe₂O₃; ⋄ La₂O₃; □ Co₃O₄; X La₂CrO₆). Weight fractions of phases and crystallite sizes were calculated from relative intensity ratio analysis and the Scherrer equation (results provided in Table 1).

Surface area analysis was performed on a Quadrasorb BET. The catalyst was pre-treated under 250° C. for 2 h, before the surface area was determined by 5 point N2 adsorption at −196° C. and the data analysed using the BET method (results provided in Table 1).

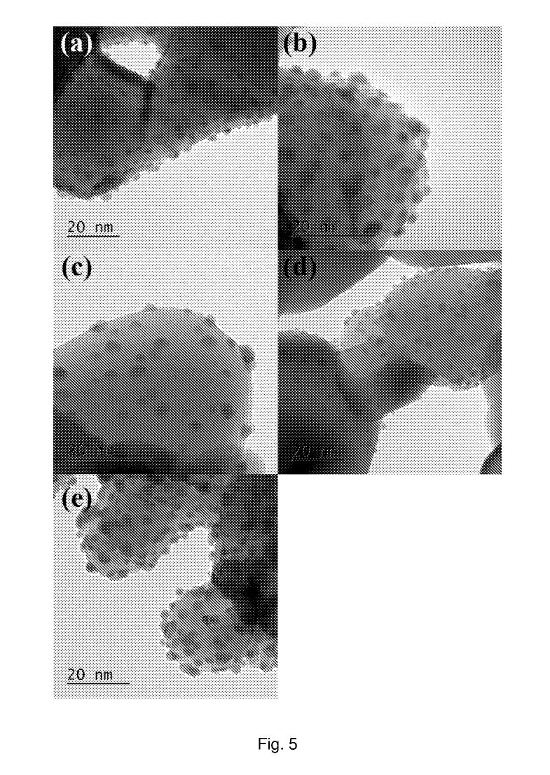

TEM was performed on the supported catalysts prepared as described above by means of a Jeol 2100 microscope with a LaB₆ filament operating at 200 kV. Samples were prepared by dispersing the powder supported catalyst in ethanol and dropping the suspension onto a lacey carbon film over a 300 mesh copper grid (representative transmission electron micrographs (TEMs) are shown in FIGS. 5a-e where the supported catalyst shown in each representative TEM is as follows: FIG. 5a AuPt/LaCrO₃; FIG. 5b AuPt/LaMnO₃; FIG. 5c AuPt/LaFeO₃; FIG. 5d AuPt/LaCoO₃; and FIG. 5e AuPt/LaNiO₃).

XPS was performed on the supported catalysts using a Kratos Axis Ultra DLD system with a monochromatic Al K_{α} X-ray source operating at 120 W. Data was collected in the hybrid mode of operation, using a combination of magnetic and electrostatic lenses, at pass energies of 40 and 160 eV for high resolution and survey spectra, respectively. The results are shown Table 2 below.

Glycerol Oxidation Testing and Product Analysis

Catalyst testing was performed using a 50 mL Radleys glass reactor. The aqueous glycerol (or glyceric acid) solution (0.3 M, containing NaOH (NaOH/glycerol ratio=4, mol/mol)) was added into the reactor. The reactor was then heated to 80° C. prior to being purged three times with oxygen. Following this the desired amount of catalyst (glycerol/metal ratio=1000, mol/mol) was suspended in the solution and the reactor heated to 100° C. The system was then pressurised to 3 bar O₂ and the reaction mixture stirred at 900 rpm. After the stated reaction time, the reactor vessel was cooled to room temperature and the reaction mixture diluted by a factor of 10 before being analysed by HPLC (Agilent 1260 infinity HPLC) equipped with ultraviolet and refractive index detectors and a Metacarb 67H column (held at 50° C.). The eluent was an aqueous solution of H₃PO₄ (0.01 M), used at a flow rate of 0.8 ml min⁻¹. Quantification of reactants consumed and products generated was determined by an external calibration method (4 concentrations, within the concentration range of the potential concentration of a product, were injected into the HPLC. The peak area was plotted against concentration to give a straight line through the origin of which the gradient is the response factor, used to normalise the area of each of the products to determine selectivity from the normalised peak area of each product). The reaction effluent was analysed for the following products; glyceric acid, tartronic acid, oxalic acid, glycolic acid, formic acid, acetic acid and lactic acid.

Results

Properties of the SAS Precipitated Perovskites

As observed in Table 1, the precipitation of near stoichiometric La and B elements from the SAS precipitations was achieved for all the different perovskites, which is an important factor when producing perovskite materials with high phase purity. However, in some cases this required an excess of the B site metal salt in the metal salt solution, to prevent excess La in the final precipitate. Non-stoichiometric precipitation was due to the different precipitation yields of the individual metal acetate salts, dictated primarily by the solubility of the salts in supercritical CO₂-methanol under the conditions used. Yields from the SAS process could be altered to give precipitate ratios closer to 1:1 from initial 1:1 starting solutions by varying the pressure, solvent: CO₂ ratio and also the solution injection geometry.

TABLE 1
Metal salts used for SAS precipitations and physical properties of the resultant perovskites

| | | Precursor | | | | | |
|---|---|---|---|---|---|---|---|
| | | solution | Precipitated | Phase | | | |
| | | La:B molar | La:B molar | composition | | Crystallite | Surface |
| Sample | B metal salt | ratio | ratio* | from XRD | | size (nm) | area (g m⁻²) |
| LaCrO₃ | Chromium | 1:1 | 1:1.06 | LaCrO₃, | | 6 | 52 |
| | (III) acetate* | | | trace La₂CrO₆* | | | |
| LaMnO₃ | Manganese | 1:1.2 | 1:1.03 | LaMnO₃ | (100%), | 18 | 32 |
| | (II) acetate | | | | | | |
| | tetrahydrate | | | | | | |
| LaFeO₃ | Iron (II) | 1:1.4 | 1:0.99 | LaFeO₃ | (85%) | 21 | 26 |
| | acetate | | | La₂O₃ | (9%) | | |
| | | | | Fe₂O₃ | (6%) | | |
| LaCoO₃ | Cobalt (II) | 1:1.1 | 1:1.05 | LaCoO₃ | (90%) | 22 | 22 |
| | acetate | | | Co₃O₄ | (10%) | | |
| | tetrahydrate | | | | | | |
| LaNiO₃ | Nickel (II) | 1:1.1 | 1:1.07 | LaNiO₃ | (90%) | 15 | 36 |
| | acetate | | | La₂O₃ | (8%) | | |
| | tetrahydrate | | | NiO | (2%) | | |

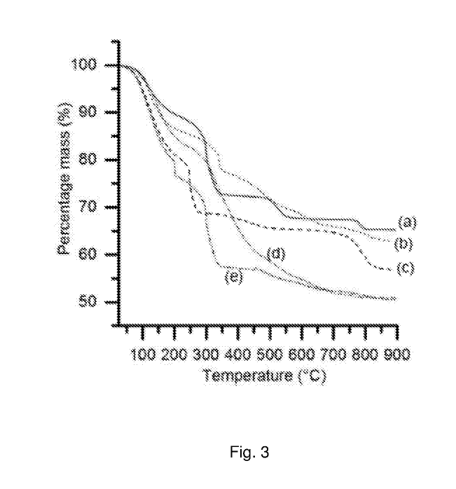

Thermogravimetric analysis (TGA) was carried out on each of the SAS precipitated materials. The results are shown in the graph of FIG. 3 in which lines (a)-(e) show the results for materials in which the B-site species was (a) Cr, (b) Fe, (c) Mn, (d) Co and (e) Ni. It has previously been shown that the SAS precipitation of Ce, Mn, Fe, Co and Ni acetate salts results in an acetate salt being retained. However, the local co-ordination geometry around metal is altered and the sample no longer displays long range order according to XRD analysis. This results in their thermal decomposition at temperatures below 400° C. to form their corresponding oxides. It is, therefore, likely that the mass losses observed up to about 450° C. are indicative of the decomposition of acetate species, with higher temperature mass losses being associated with the transitions between various metal oxide and mixed metal oxide phases. The number of ternary oxide permutations is dependent on the ability of the B site element to adopt different valence states. These ternary phases, decompose at specific temperatures to produce the perovskite phase and O₂. Using the data obtained in the TGA analysis, the SAS precipitated materials were calcined at 750° C., as TGA showed the final mass loss event had started before this temperature.

XRD analysis (FIG. 4 and Table 1) of the calcined materials showed that perovskite phases were dominant. Smaller amounts of by-product phases were also observed.

The average crystallite sizes of the SAS precipitated perovskites were calculated from the Scherrer equation using the XRD patterns, and were found to be between 6 and 22 nm (Table 1). Relative to perovskites prepared by more conventional methods, the crystallite sizes observed from SAS precipitation were relatively small. The small particle size of the SAS precipitated perovskites resulted in surface areas in the region of 22-52 m² g⁻¹, which are greater than the 1-15 m² g⁻¹ found for perovskites prepared by more conventional techniques. The combination of high surface area and small crystallite size is thought to provide a suitable number of surface sites for the anchoring of metal/metal alloy catalytic particles (e.g. metal/metal alloy nanoparticles).

The sol immobilisation technique described above for depositing catalytic particles on the perovskite support, using PVA as the protecting ligand, was used to deposit 1 wt % AuPt (1:1 molar ratio) nanoparticles onto the perovskite supports. For all perovskite supports the desired metal content and Au:Pt ratio was deposited (calculated from MP-AES data shown in Table 2).

TABLE 2
Au and Pt surface and bulk composition of
AuPt/perovskite catalysts from MP-AES and XPS analysis

| | Au and Pt content | Bulk Au/Pt ratio | Surface Au/Pt ratio |
|---|---|---|---|
| Support | (wt. %) | from MP-AES | from XPS |
| LaCrO₃ | 0.55 (Au), 0.55 (Pt) | 1.0 | 0.6 |
| LaMnO₃ | 0.47 (Au), 0.46 (Pt) | 1.0 | 1.4 |
| LaFeO₃ | 0.46 (Au), 0.47 (Pt) | 1.0 | 1.0 |
| LaCoO₃ | 0.55 (Au), 0.54 (Pt) | 1.0 | 0.6 |
| LaNiO₃ | 0.50 (Au), 0.48 (Pt) | 1.1 | 0.9 |

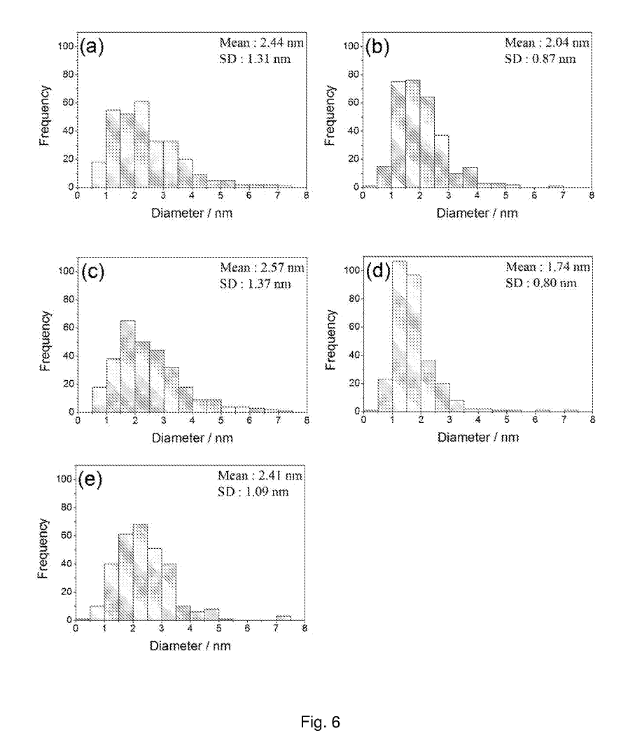

Representative TEM images, with corresponding particle size distributions of the AuPt catalytic particles, are shown in FIGS. 5a-e (representative TEMs for the following supported catalysts: (a) AuPt/LaCrO₃; (b) AuPt/LaMnO₃; (c) AuPt/LaFeO₃; (d) AuPt/LaCoO₃; and (e) AuPt/LaNiO₃.) and FIG. 6a-e (Particle size distribution histograms of AuPt supported on the different SAS prepared LaBO₃ perovskites. (a) AuPt/LaCrO₃; (b) AuPt/LaMnO₃; (c) AuPt/LaFeO₃; (d) AuPt/LaCoO₃; (e) AuPt/LaNiO₃) respectively. In all cases a mean particle size (calculated from the particle size of about 500 particles measured using TEM) of ca. 2 nm with a standard deviation of ca. 1 nm was observed. The slight size variation in the metal nanoparticle size between the different perovskite supported catalysts was found to have no strong correlation with either the surface area or the B site element. The perovskites prepared by the antisolvent precipitation methodology were found to have sufficient surface area to successfully support 1 wt % AuPt.

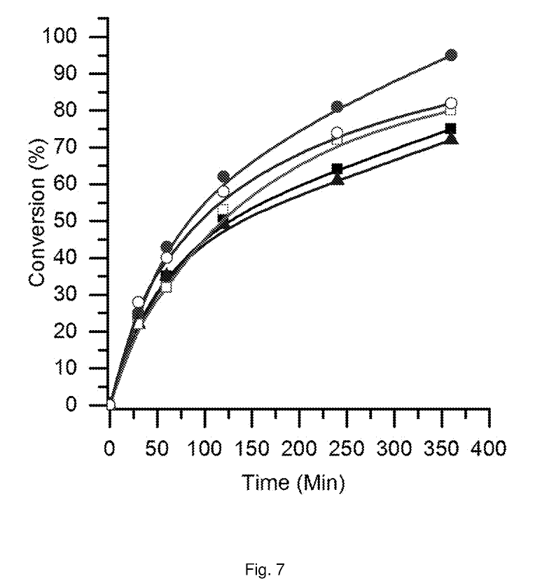

The effect of the B site in AuPt/perovskite catalysts for the glycerol oxidation reaction (Conditions; Glycerol 0.3 M in water, 4:1 NaOH:glycerol, metal:glycerol=1000, 3 bar O₂, temperature=100° C.) was investigated, with conversion profiles shown in FIG. 7 (The conversion of glycerol with AuPt/LaBO₃ catalysts, where the B sites of the supports are; Cr (●); Mn (▪); Fe (▴); Co (□); Ni (◯)) and TOFs (mol_{glycerol converted} mol_{AuPt}⁻¹ h⁻¹) given in Table 3.

TABLE 3
Glycerol oxidation using AuPt supported on the
perovskite and single oxide materials

| Supported | | Selectivity^{b} (%) | | | |
|---|---|---|---|---|---|
| Catalyst | TOF (h⁻¹)^{a} | Glyc | Tar | C-C scission | Lac |
| AuPt/LaCrO₃ | 620 | 5 | 7 | 2 | 86 |
| AuPt/LaMnO₃ | 460 | 87 | 0 | 13 | 0 |
| AuPt/MnO₂ | 560 | 33 | 3 | 33 | 31 |
| AuPt/LaFeO₃ | 440 | 10 | 2 | 19 | 69 |
| AuPt/Fe₂O₃ | 240 | 19 | 2 | 58 | 21 |
| AuPt/LaCoO₃ | 440 | 43 | 9 | 24 | 24 |
| AuPt/Co₃O₄ | 180 | 23 | 4 | 24 | 49 |
| AuPt/LaNiO₃ | 560 | 30 | 3 | 26 | 41 |
| AuPt/NiO | 700 | 30 | 10 | 39 | 21 |

^{a}TOF calculated at 30 min, moles of glycerol converted/moles of metal/h.
^{b}Selectivity calculated after 6 h reaction

It can be seen that all catalysts had similar initial rates, with the TOF of the AuPt/LaCrO₃ and AuPt/LaNiO₃ catalysts being slightly higher at 620 h⁻¹ and 560 h⁻¹, compared with the other catalysts which had TOFs of 440-460 h⁻¹. No correlation between the TOF and the AuPt nanoparticle size was observed, although this was expected as the variance in TOFs and particle sizes was small.

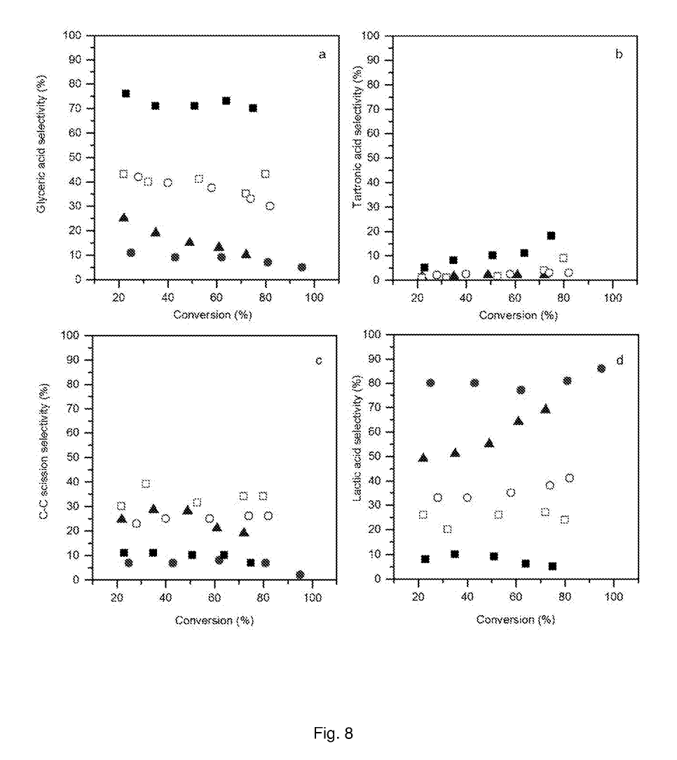

The product selectivity with different AuPt/perovskite supported catalysts is shown in FIG. 8 (the B sites of the perovskite supports of the supported catalysts used and results shown in FIG. 8 are; Cr (●); Mn (▪); Fe (▴); Co (□); Ni (◯)). The activities of the different supported catalysts were very similar, whereas, the product distributions were markedly different. The LaMnO₃ supported catalyst was found to favour C₃ oxidation products, with high selectivity to glyceric acid, which at high conversions further oxidised to tartronic acid. Selectivities to C—C scission products (oxalic acid, glycolic acid, formic acid and CO₂) and lactic acid were low and consistent across the range of conversions observed for the LaMnO₃ supported catalyst. This is an interesting result as the reaction conditions used are reported to enhance the dehydration and re-arrangement of glyceraldehyde to lactic acid. Under these relatively high temperatures and high base concentrations, AuPt nanoparticles supported on CeO₂ or TiO₂ have been reported to give lactic acid selectivities between 60 and 80%. It is apparent that employing the LaMnO₃ support switches off the lactic acid pathway and promotes the oxidation pathway.

The selectivity profiles for the LaCoO₃ and LaNiO₃ supported catalysts are similar, with moderate selectivity to glyceric acid, relatively high C—C scission selectivity and lactic acid selectivity of ca. 30%. It was noted that for the AuPt/LaNiO₃ catalyst, glyceric acid selectivity decreased when glycerol conversion increased from 28% to 82%. This decrease in glyceric acid selectivity did not correspond to a further oxidation to tartronic acid, but was accompanied by an increase in lactic acid formation, indicating a change in the prevalence of the oxidation and dehydration reaction pathways.

The selectivity profile of the AuPt/LaFeO₃ also changed with respect to glycerol conversion. At low conversions the AuPt/LaFeO₃ catalyst had a glyceric acid selectivity of 25%, scission products of 24.5% and 49% selectivity to lactic acid. As the reaction progressed and glycerol conversion increased, the selectivity towards the glyceric acid decreased dramatically. As observed with the AuPt/LaNiO₃ catalyst, the change in the selectivity profile was the result in a shift towards lactic acid production, with the selectivity to this product increasing from 49% to 69% over the reaction period. The shift towards lactic acid formation was far more significant in the LaFeO₃ supported catalyst than any of the other perovskite supported catalysts. This may suggest that the oxidation sites on this catalyst are not stable or are blocked by reaction intermediates.

The highest lactic acid yield was observed with the AuPt/LaCrO₃ catalyst, with 86% selectivity to this product at 95% glycerol conversion. Unlike the LaFeO₃ supported catalyst, selectivity towards lactic acid was relatively insensitive to conversion, with only a slight increase in lactic acid selectivity from 80% to 86% over the full conversion range. With a TOF of 620 h⁻¹ and selectivity towards lactic acid above 85%, the AuPt/LaCrO₃ catalyst is a highly effective catalyst for lactic acid production from glycerol.

Evidently the variation of the B-site species of the perovskite support of the supported catalyst had a dramatic effect on the glycerol oxidation product distribution. The choice of Mn for the B site resulted in suppression of the lactic acid pathway with glyceric acid being the dominant product, with a Cr or Fe B sites the dehydration pathway to lactic acid is promoted, and a Ni or Co B site produces both oxidation and dehydration products. Both the lactic acid pathway and oxidation pathway to glyceric or tartronic acid proceed via the glyceraldehyde intermediate. As the rate limiting step for both reaction pathways is the initial proton abstraction from glycerol to form the alkoxy intermediate, substantially different product distributions were observed alongside very similar catalyst activities.

Therefore, the present inventors have found that varying the B-site species in a perovskite support (e.g. a support that is inactive or unselective in the absence of a catalytic component) allows the selectivity a supported catalyst comprising the perovskite support to be tuned.

The present inventors also tested whether any single metal oxide phases present were responsible for differences in product selectivity, AuPt was deposited on MnO₂, Fe₂O₃, Co₃O₄ and NiO supports prepared by the SAS process (calcined at 750° C.) and tested as catalysts for the glycerol oxidation reaction (see Table 3). Unlike the corresponding perovskite supported catalysts, the single oxide supported catalysts had a significant range of TOFs from 180 to 700 h⁻¹, with the TOF for the Ni and Mn single oxides being higher than their corresponding perovskite catalysts. It is important to note that all of the catalysts were less selective than the AuPt/perovskites, with the highest selectivity to any product being 49% lactic acid with the AuPt/Co₃O₄. Specifically, the high selectivity towards glyceric acid with AuPt/LaMnO₃ catalyst (69%) was not replicated with the AuPt/MnO₂ (33% glyceric acid selectivity) or the high lactic acid selectivity in AuPt/LaFeO₃ compared to the AuPt/Fe₂O₃ (69% vs 21%).

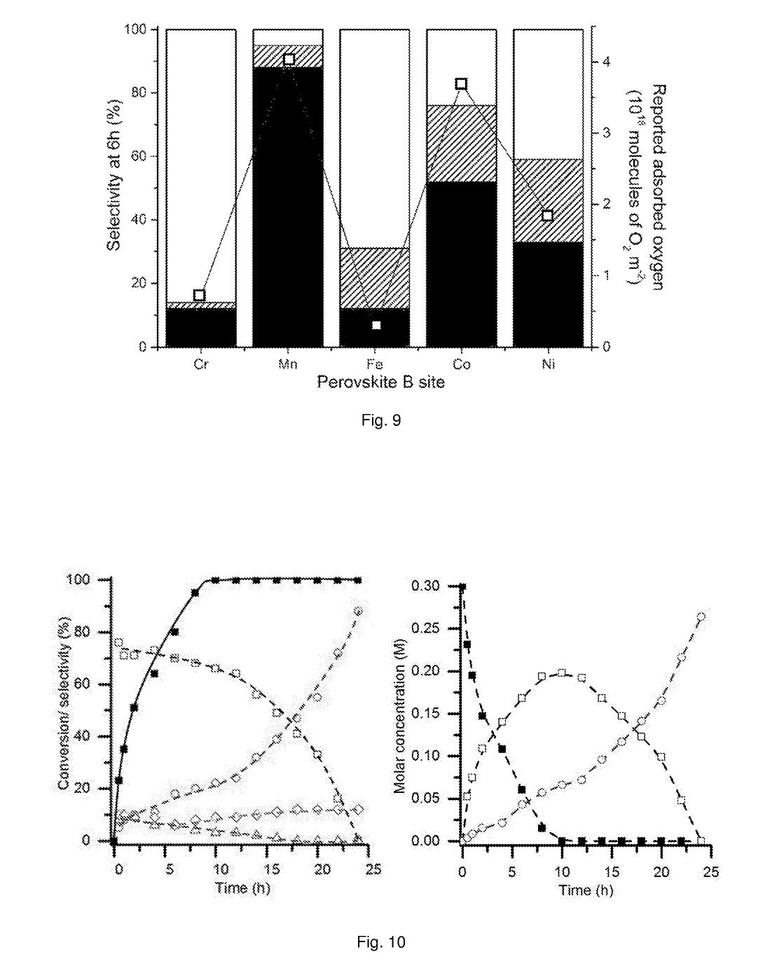

Perovskites have been extensively researched as catalysts for the deep oxidation of alkanes, alkenes and CO (G. Kremenic, J. M. L. Nieto, J. M. D. Tascon and L. G. Tejuca, Journal of the Chemical Society, Faraday Transactions 1: Physical Chemistry in Condensed Phases, 1985, 81, 939-949 and L. G. Tejuca, J. L. G. Fierro and J. M. D. Tascon, Adv. Catal., 1989, 36, 237-328, 385-236). A strong correlation between activity and O₂ coverage profiles have been reported, with perovskites with good O₂ absorption capacities being more active. Tejuca et al. investigated the chemisorption of O₂ and isobutene on a range of LaBO₃ catalysts with the same range of B sites used in this study (i.e Cr, Mn, Fe, Co and Ni). The adsorption profiles of O₂ on the LaBO₃ clean surfaces from this earlier study have been plotted against the glycerol oxidation selectivity profiles of the various B sites (at 6 h reaction time) used in the AuPt/LaBO₃ (see FIG. 9 which shows selectivity profiles of the AuPt/LaBO₃ catalysts compared to reported oxygen adsorption values (□) for the relevant perovskite phases (see G. Kremenic, J. M. L. Nieto, J. M. D. Tascon and L. G. Tejuca, Journal of the Chemical Society, Faraday Transactions 1: Physical Chemistry in Condensed Phases, 1985, 81, 939-949). The sum of C3 oxidation products (glyceric and tartronic acid) shown in solid black. C—C scission products (sum of glycolic acid, oxalic acid, formic acid and CO₂) shown in cross-hatched and lactic acid selectivity shown by the solid white bar. If C—C scission products are assumed to be produced from an oxidation process, the sum of the oxidation pathway products correlate well with the reported oxygen adsorption capacities. The most selective catalyst towards the oxidation pathway used the LaMnO₃ support, which had the best oxygen adsorption capacity. The LaCrO₃ and LaFeO₃ supports with poor oxygen adsorption characteristics were found to give catalysts that favour the production of lactic acid, which is formed from an initial oxidation followed by dehydration to pyruvaldehyde and rearrangement. The LaCoO₃ and LaNiO₃ supported catalysts were found to produce both oxidation and the dehydration products that correspond to the intermediate oxygen adsorption capacities.

The activities of the perovskite supports in the absence of a catalytic component were tested under the conditions described above for glycerol oxidation. The perovskite supports were found to be not active.

As the glycerol conversion did not reach 100% over the 6 h reaction time using the AuPt/LaMnO₃ catalyst, an experiment with the reaction time extended to 24 h was performed (Conditions; Glycerol 0.3 M in water, 4:1 NaOH:glycerol, metal:glycerol=1000, 3 bar O₂, temperature=100° C.). The conversion, selectivity profile and molar concentrations with respect to reaction time are shown in FIG. 10 (FIG. 10 provides graphs showing time on line conversion and selectivity (left) and time on line molar concentration (right) plots for extended glycerol oxidation reaction time using a AuPt/LaMnO₃ supported catalyst with the products indicated as follows: ▪ glycerol; □ glyceric acid, ◯ tartronic acid, ⋄ C—C scission A lactic acid). 100% conversion was observed after 10 h time on line, at which point selectivity towards glyceric acid was 66% with tartronic acid selectivity of 22%. This represents a slight increase in tartronic acid selectivity from the 18% observed at 6 h reaction time. After all the glycerol had been converted it can be seen from the molar concentration plot that glyceric acid is being converted to tartronic acid. Under these reaction conditions it was clear that the sequential oxidation of glycerol to tartronic acid, via glyceric acid, takes place. After 24 h reaction time all of the glyceric acid had been converted to give a final selectivity to tartronic acid of 88%, with C—C scission accounting for the remaining products. A yield of 88% tartronic acid in the context of the general literature is exceptionally high, it can be seen that the AuPt/LaMnO₃ supported catalyst suppresses the dehydration route to lactic acid and excessive C—C scission.

An important consideration in the application of heterogeneous catalysts is their reusability and resistance to leaching. Effluent analysis from reactions (Reaction conditions: Glycerol 0.3 M in water, 4:1 NaOH:glycerol, metal:glycerol=1000, 3 bar O₂, 100° C., 6 h) using AuPt/LaMnO₃ and AuPt/LaFeO₃ by MP-AES is shown in Table 4.

TABLE 4
Effluent analysis foliowing glycerol oxidation reaction

| | La in effluent | B element in | % La | % B site |
|---|---|---|---|---|
| Catalyst | (ppm) | effluent (ppm) | leaching | leaching |
| AuPt/LaMnO₃ | 58 | 20 | 2.1 | 1.1 |
| AuPt/LaFeO₃ | 168 | 36 | 6.0 | 1.9 |

These two catalysts were chosen for their different selectivity profiles and also in the case of the LaFeO₃ sample due to the notable changes in selectivity during the reaction. In respect to the B site leaching less than 2% of the possible metal was found in either reaction effluent. This level of leaching would not contribute to the reaction, as shown by the absence of activity for the perovskite catalysts tested without AuPt. Slightly higher levels of La leaching were determined (between 2-6% of the total La), although again this had little effect on the reaction.

To study the reusability of the catalyst, sequential reactions were performed with the supported catalyst washed with water, filtered and oven dried (120° C., 16 h) between reactions. Re-use of the AuPt/LaMnO₃ catalyst was tested over multiple 6 h reactions. Glycerol conversion was noted to increase over the 1^{st} and 2^{nd} re-use tests with the selectivity towards glyceric acid and tartronic acid remaining constant. This slight increase in conversion can be attributed to the removal of the PVA protecting agent under reaction conditions, exposing more active metal surface area.

The LaMnO₃ support was prepared by two alternative routes to the SAS precipitation method described above, mechanochemical synthesis from the single metal oxides (The milling procedure used a planetary ball mill (Retsch PM100). La₂O₃ and Mn₂O₃ were added to a ZrO₂ milling vessel with six 15 mm ZrO₂ balls before being ground at 700 rpm for 16 h. The resulting dry powder was recovered and calcined in static air at 700° C. for 4 h.) and the flame pyrolysis of metal nitrate solutions (Aqueous La/B nitrate solutions (0.1 M) were sprayed at a rate of 0.5 ml/min via a Sonozap ultrasonic nebuliser (2.8 W, 130 kHz) into a horizontally aligned propane (0.5 L/min) and oxygen (1.4 L/min) flame (0.082″ diameter stainless steel nozzle). Gas flows were controlled using mass flow controllers. The resulting powder was collected on a water cooled quartz plate 10 cm from the nozzle tip. Typical collection time was 10 minutes), and 1.wt % AuPt nanoparticles were deposited on the supports as described above. It was found that, although the mechanochemical synthesis resulted in a low surface area perovskite support which resulted in low conversion of glycerol, all the catalysts tested had the same selectivity towards the C₃ oxidation products (glyceric and tartronic acid). From an applied perspective this is a key finding, as it illustrates that the phenomenon of LaMnO₃ supports inhibiting lactic acid production is not unique to any specific preparation technique. Provided a LaMnO₃ support with sufficient surface area can be produced, a catalyst with strong selective oxidation potential can be synthesised.

Therefore, the present inventors have demonstrated that the selectivity of a supported catalyst can be tuned by varying the B-site species however the perovskite support is produced.

1. A method for making a desired reaction product under liquid-phase conditions, the method comprising:
providing a supported catalyst having selectivity for the desired reaction product, the supported catalyst comprising:
a perovskite support comprising an A-site species and a B-site species; and
metal or metal alloy catalytic particles on a surface of the perovskite support, wherein the B-site species is selected to provide selectivity for the desired reaction product; and
contacting reactants with the supported catalyst to provide the desired reaction product.

2. A method according to claim 1, wherein the reactants and supported catalyst are contacted at a temperature of less than 150° C.

3. A method according to claim 1, wherein the reactants comprise glycerol and oxygen, and the B-site species is selected such that the supported catalyst has selectivity for the oxidation of glycerol.

4. A method according to claim 3, wherein the desired reaction product is glyceric acid, tartronic acid or lactic acid.

5. A process for tuning the selectivity of a supported catalyst comprising a perovskite support comprising an A-site species and a B-site species, and metal or metal alloy catalytic particles deposited on the perovskite support, the process comprising varying the B-site species of the perovskite support to tune the selectivity of the supported catalyst.

6. A process according to claim 5, wherein the B-site species of the perovskite support is varied while the A-site species of the perovskite support and the metal or metal catalytic particles on the perovskite support are unchanged.

7. A process for identifying a supported catalyst having selectivity for a desired reaction product, the process comprising:
(a) selecting a reaction for producing the desired reaction product;
(b) selecting a metal or metal alloy for catalysing the selected reaction;
(c) providing a plurality of supported catalysts, each supported catalyst comprising:
a perovskite support comprising an A-site species and a B-site species; and
catalytic particles of the selected metal or metal alloy on a surface of the perovskite support,
each of the supported catalysts having a different B-site species;
(d) carrying out the selected reaction using each of the supported catalysts provided in step (c); and
(e) determining the selectivity of each of the supported catalysts provided in step (c) for the desired reaction product.

8. A process according to claim 7, wherein each supported catalyst comprises the same A-site species selected from the group comprising alkaline earth metal, lanthanide cations and combinations thereof.

9. A process according to claim 5, wherein the B-site species is selected from the group comprising transitional metal cations and combinations thereof.

10. A process according to claim 5, wherein the or each supported catalyst contains at least about 0.5 wt. % metal or metal alloy catalytic particles by total weight of the supported catalyst.

11. A process according to claim 5, wherein the perovskite support of the supported catalyst is inactive or provides no selectivity towards the desired reaction product in the absence of the metal or metal alloy catalytic particles.

12. A process according to claim 5, wherein the perovskite support has a BET surface area of greater than about 15 m²/g.

13. A process according to claim 5, wherein the supported catalyst has a crystallite size of less than about 50 nm.

14. A method of forming a supported catalyst for a liquid-phase reaction, the method comprising:
providing a perovskite support comprising an A-site species and a B-site species, wherein the B-site species is selected to control the selectivity of the supported catalyst towards a desired reaction product;
depositing metal or metal alloy catalytic particles on a surface of the perovskite support; and
exposing the supported catalyst to a temperature not greater than about 350° C. such that the metal or metal alloy catalytic particles remain on the surface of the perovskite support.

15. A method according to claim 14, wherein depositing the metal or metal alloy catalytic particles on the surface of the perovskite support comprises impregnating the perovskite support with an aqueous solution containing ions of the metal or metal alloy.

16. A perovskite supported catalyst for a liquid-phase reaction, the supported catalyst comprising:
a perovskite support comprising an A-site species and a B-site species; and
metal or metal alloy catalytic particles on a surface of the perovskite support.

17. A supported catalyst according to claim 16, the perovskite support having a BET surface area of greater than about 20 m²/g.

18. A supported catalyst according to claim 16, wherein the supported catalyst has a crystallite size of less than about 50 nm.

19. A supported catalyst according to claim 16, wherein the B-site species provides selectivity of the supported catalyst for a desired reaction product.

20. A supported catalyst according to claim 16, wherein the A-site species is selected from the group comprising alkaline earth metal cations and lanthanide cations.

21. A supported catalyst according to claim 16, wherein the B-site species is selected from transition metal cations.

22. A supported catalyst according to claim 16, wherein the A-site species is lanthanum, the B-site species is manganese, and the nanoparticles comprise gold and platinum.

23. A supported catalyst according to claim 16, wherein the A-site species is lanthanum, the B-site species is selected from iron and chromium, and the nanoparticles comprise gold and platinum.

24-26. (canceled)
